(19) United States
(12) Patent Application Publication
(10) Pub. No.: US 2025/0015269 A1
(43) Pub. Date: Jan. 9, 2025

(54) HYBRID GRAPHENE ELECTRODE

(71) Applicant: NANOGENESIS INC., Seoul (KR)

(72) Inventor: Joon Sub SHIM, Yongin-si (KR)

(21) Appl. No.: 18/711,096

(22) PCT Filed: Nov. 17, 2021
(86) PCT No.: PCT/KR2021/016913

§ 371 Date: May 16, 2024

(30) Foreign Application Priority Data
Nov. 17, 2021 (KR) 10-2021-0158569

(51) Int. Cl. H01M 4/36; H01M 4/02; H01M 4/04; H01M 4/38; H01M 4/583; H01M 10/052

(52) U.S. Cl. CPC H01M 4/366; H01M 4/0471; H01M 4/382; H01M 4/583; H01M 10/052; H01M 2004/025; H01M 2220/30

(57) ABSTRACT

Provided is a hybrid graphene electrode provided with a graphene composite having a structure in which a plurality of micro particles and multi-layered graphene are mixed, wherein the micro particles are metal or semiconductor particles and adhere to the surface or inside of the multi-layered graphene, some of the micro particles bond and coagulate with each other, the multi-layered graphene has a three-dimensional structure in which several layers of graphene are laminated and bent in an arbitrary direction, a portion of empty spaces between the micro particles is filled with the multi-layered graphene, thus forming an interconnected structure, and electrons flow through the graphene composite.

### TECHNICAL FIELD

The present invention relates to a hybrid graphene electrode, and more particularly, to a specific antigen hybrid graphene electrode which has excellent selectivity and specificity to a low-concentration antigen, and particularly, is optimized for an immune sensor for detection of dementia-specific antigens by using formation of a 3-dimensional structure by cross-linking of micro particles and graphene and high electrical conductivity characteristics by such a structural feature.

### BACKGROUND ART

Graphene is a material that not only has very stable and excellent electrical, mechanical, and chemical properties, but also has excellent conductivity and a material that moves electrons about 100 times faster than silicon and allows about 100 times more current to flow than copper, and research on preparation and application thereof has been actively conducted.

This biosensor applied with the graphene may be used as an immune sensor based on antigen-antibody binding. Like a biomarker, the immune sensor has been widely used to detect disease-related materials in clinical diagnosis. due to the specific binding of an antibody against the antigen, the antibody is fixed onto the surface of the immune sensor, and used particularly to detect the biomarker.

For example, as a prostate cancer marker, prostate specific antigen (PSA) is widely used for screening, diagnosis, and treatment of prostate cancer. The prostate specific antigen is an enzyme synthesized and secreted by the epithelial cells of the prostate, and in general people, the concentration is measured at 0 to 4 ng/ml, but in prostate cancer patients, the concentration is measured to be higher. Therefore, immune sensors with excellent selectivity, specificity, and sensitivity to prostate-specific antigens may be useful for early diagnosis and prevention of prostate cancer.

The immune sensor includes two types of sandwich-type immune sensor and label-free immune sensor. In the sandwich type, a primary antibody capable of binding to the antigen is immobilized on the surface of a substrate, and a labeled antibody capable of binding to a prostate-specific antigen is used as a secondary antibody. The sandwich type uses the primary and labeled secondary antibodies to obtain antigen-antibody binding efficiency, selectivity, sensitivity, and signal amplification effects. Unlike this, the label-free immune sensor is a notable biomarker detection and analysis tool that not only has excellent convenience, speed, and sensitivity, but also has excellent economic efficiency due to reduced costs by directly measuring antigen-antibody binding.

For the development of better label-free immune sensors, graphene-based composites using graphene, which has excellent biocompatibility and electron transfer properties, are attracting attention as electrode materials. Therefore, research on the application of graphene to biosensors and the like has been actively conducted, and it is known that graphene may effectively contribute to the development of electrochemical biosensors with extremely high sensitivity.

Korean Patent Registration No. 1400976 discloses a biosensor that connects a molecular linker to a reduced graphene oxide layer and adds a metal nano particle layer, but has a horizontal structure, not a three-dimensional structure, and has limited molecular linkers, and Korean Patent Registration No. 1339403 discloses a reduced graphene oxide-metal nano particle composite film, but only the possibility of using the micro particle composite film as a biosensor has been suggested.

Accordingly, the present inventor developed a three-dimensional microparticle-graphene composite manufactured using photochemical and photothermal irradiation, and an immune sensor based thereon has the characteristics of high sensitivity and excellent reproducibility as well as selectivity, specificity, and economic efficiency. The present invention may be particularly applied to an immune sensor that is very effective in detecting dementia-specific antigens.

### DISCLOSURE

### Technical Problem

An object of the present invention is to provide a hybrid graphene electrode for a dementia-specific antigen immune sensor which has excellent sensitivity and specificity to a low-concentration antigen, and particularly, is optimized for an immune sensor for detection of dementia-specific antigens by using formation of a 3-dimensional structure by cross-linking of micro particles and graphene and high electrical conductivity characteristics by such a structural feature.

### Technical Solution

In order to achieve the object, the present invention provides a hybrid graphene electrode provided with a graphene composite having a structure in which a plurality of micro particles and multi-layered graphene are mixed, in which the micro particles are metal or semiconductor particles and adhere to the surface or inside of the multi-layered graphene, some of the micro particles bond and coagulate with each other, the multi-layered graphene has a three-dimensional structure in which several layers of graphene are laminated and bent in an arbitrary direction, and a portion of empty spaces between the micro particles is filled with the multi-layered graphene, thus forming an interconnected structure, and electrons flow through the graphene composite.

Further, the present invention provides the hybrid graphene electrode in which graphene is coated on the surface of the micro particles.

Further, the present invention provides the hybrid graphene electrode in which the graphene composite is generated by photochemical or photothermal irradiation or heat treatment process.

Further, the present invention provides the hybrid graphene electrode in which external electrons are introduced or released through the graphene composite.

Further, the present invention provides a hybrid graphene electrode in which the micro particles include gold (Au), silicon (Si), silicon carbide (SiCx including Si₂C, SiC, or SiC₂), silicon oxide (SiOx including SiO or SiO₂), silver (Ag), and silver (Ag) coated on a copper metal surface.

Further, the present invention provides the hybrid graphene electrode for an electrochemical sensor in which in the graphene metal composite, a specific target material is detected by using an electrochemical reaction.

Further, the present invention provides a hybrid graphene electrode in which lithium (Li) ions are bonded to and separated from the graphene composite to cause charging and discharging.

### Advantageous Effects

According to the present invention, a hybrid graphene electrode having a 3-dimensional structure by cross-linking of micro particles and graphene for a dementia-specific antigen immune sensor has excellent sensitivity to a low-concentration antigen, and particularly, is optimized for an immune sensor for detection of dementia-specific antigens by using high electrical conductivity characteristics by such a structural feature.

### DESCRIPTION OF DRAWINGS

FIGS. 1A to 1E are step-by-step SEM photographs and conceptual diagrams of a hybrid graphene electrode according to the present invention.

FIG. 2 is a graph showing comparing electrical conductivity characteristics of a hybrid graphene electrode (graphene metal composite), a metal electrode, and a graphene electrode according to the present invention.

FIG. 3 is a graph showing measured currents according to a concentration of electrochemical measuring material (PAP) of a hybrid graphene electrode, a graphene electrode, and a metal electrode according to the present invention.

FIG. 4 is a diagram showing a difference in current signal measured for PAPs of the same concentration for the graphene metal composite electrode, the graphene electrode, and the metal electrode according to the present invention.

FIG. 5 is a diagram showing an interdigitated electrode (IDA) using a hybrid graphene electrode according to the present invention.

### MODE FOR INVENTION

Hereinafter, preferred embodiments of the present invention will be described in detail. In describing the present invention, a detailed description of known functions and configurations incorporated will be omitted so as to avoid obscuring the gist of the present invention.

The terms “about”, “substantially”, and the like used in the present specification are used as a numerical value or a value close to the numerical value when inherent manufacturing and material tolerances are presented in the stated meaning, and used to prevent an unscrupulous infringer from unfairly using disclosed contents in which precise or absolute numerical values are mentioned to help in the understanding of the present invention.

The present invention relates to a hybrid graphene electrode in which a graphene composite having a mixed structure of micro particles and a graphene composite layer is configured, and a flow of electrons occurs through the graphene metal composite.

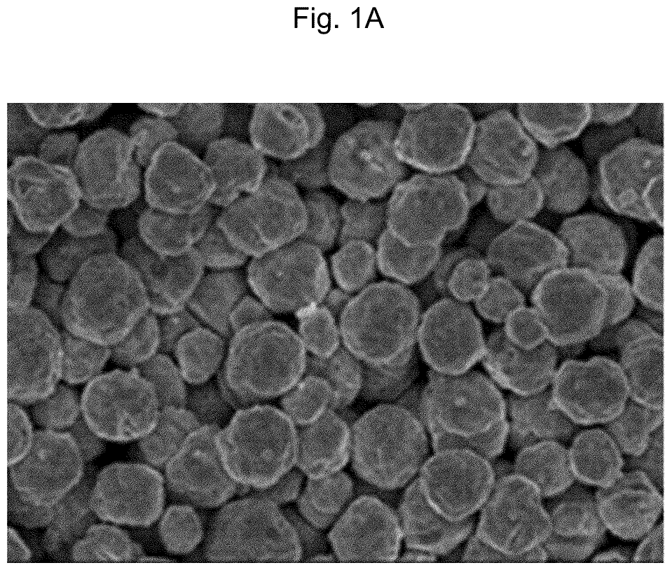

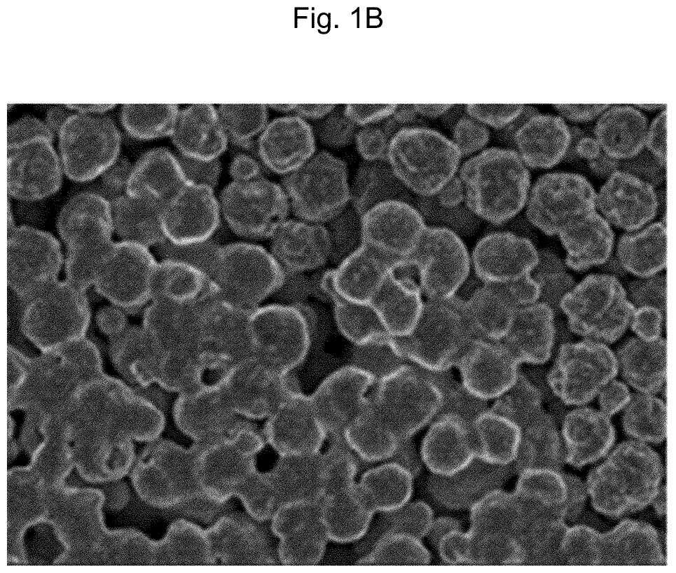

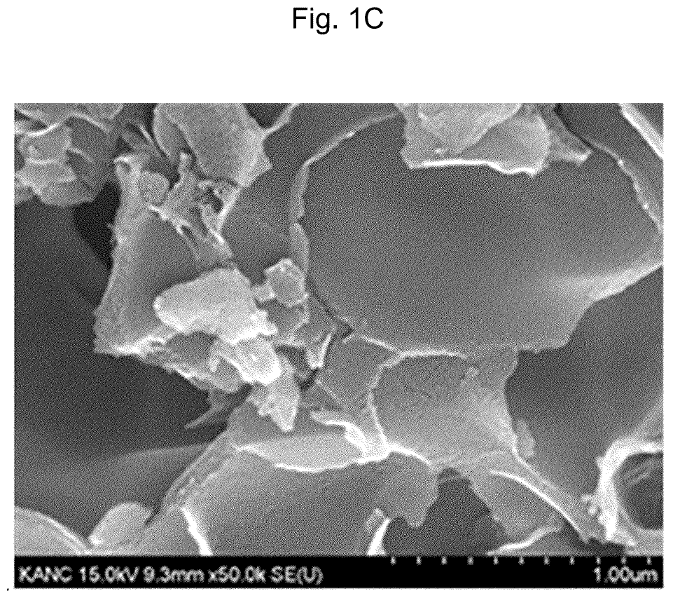

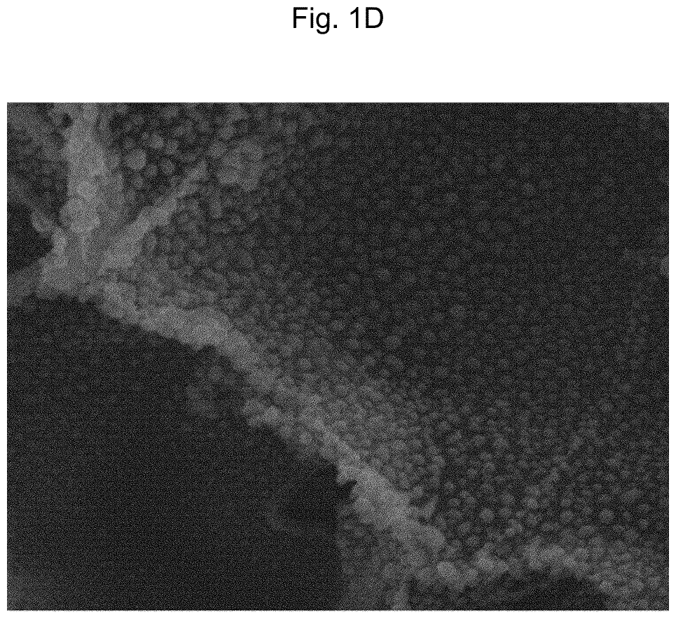

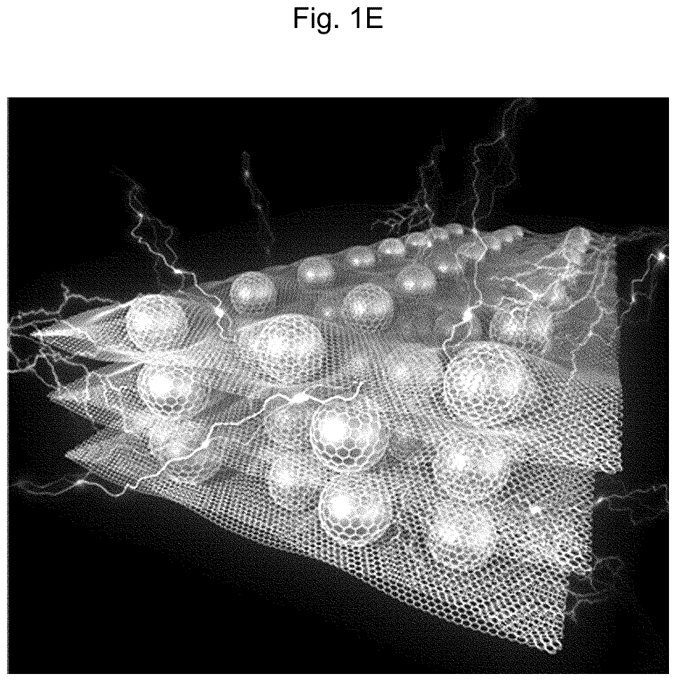

FIGS. 1A to 1E are step-by-step SEM photographs and conceptual diagrams of a hybrid graphene electrode according to the present invention. FIG. 1A is a photograph showing silver (Ag) micro particles, which have a spherical particle diameter of about 5 μm as one example of the present invention. FIG. 1B is a photograph of the surface of silver (Ag) microparticles which is bonded and coagulated with adjacent micro particles in a melted state using a photochemical, photothermal irradiation, or heat treatment process. Some micro particles are not connected to form empty spaces. FIG. 1C is a photo related to a graphene (multi-layer graphene is bent in a 3D structure) after photochemical and photothermal reactions.

Graphene is one of the allotropes of carbon and has a structure in which carbon atoms come together to form a two-dimensional plane. The respective carbon atoms form a hexagonal lattice, and the carbon atom is located at each vertex of the hexagon. At the nano size, the two-dimensional planar graphene is characterized by having an irregular shape with an overlapping or bent structure.

The micro particles include gold (Au), silicon (Si), silicon carbide (SiCx including Si₂C, SiC, or SiC₂), silicon oxide (SiOx including SiO or SiO₂), silver (Ag), and silver (Ag) coated on a copper metal surface.

In addition, a semiconductor particle is a particle that corresponds to a semiconductor material, and as a material having an electrical conductivity which is intermediate between that of a conductor such as copper and an insulator (nonconductor) such as glass includes all materials having a property in which a conductivity is changed when a voltage or wavelength of heat or light is applied to a semiconductor. The semiconductor particle primarily refers to a silicon (Si) particle which is a unique semiconductor. Besides, non-unique semiconductors include a mixture of phosphorus (P), arsenic (As), antimony (Sb), bismuth (Bi), boron (B), aluminum (Al), indium (In), and gallium (Ga).

When the graphene composite layer is formed of graphene and semiconductor silicon particles, the graphene composite layer is used as a battery anode material in which charging and discharging occur through a process in which lithium ions are bonded to and separated from the graphene and silicon particles.

FIG. 1D is the SEM photo of the hybrid graphene electrode of the present invention, which shows a fixed structure in which the micro particles may be bound to the surface or the inside of the graphene composite layer and some of the micro particles may bind and coagulate with each other by a photochemical or photothermal irradiation.

FIG. 1E is a conceptual diagram showing the structure in which graphene prepared by the photochemical or photothermal reaction of FIG. 1D is located and fixed in an empty space (FIG. 1B) of silver (Ag) micro particles. The silver (Ag) micro particles may be bound to the inside or the outside of the graphene composite layer, and although not shown in the conceptual diagram, some micro particles may be mutually bonded and coagulated according to irregular locations of the silver (Ag) micro particles.

Additionally, a graphene coating may be generated on the surface of the silver (Ag) micro particles through the photochemical or photothermal reaction. In FIG. 1E, a graphene coating structure is shown as a mesh on the surface of the particle.

Existing graphene requires complex processes, including A high-temperature process, but a photothermal or photochemical synthetic graphene may be synthesized relatively simply through a one-step process.

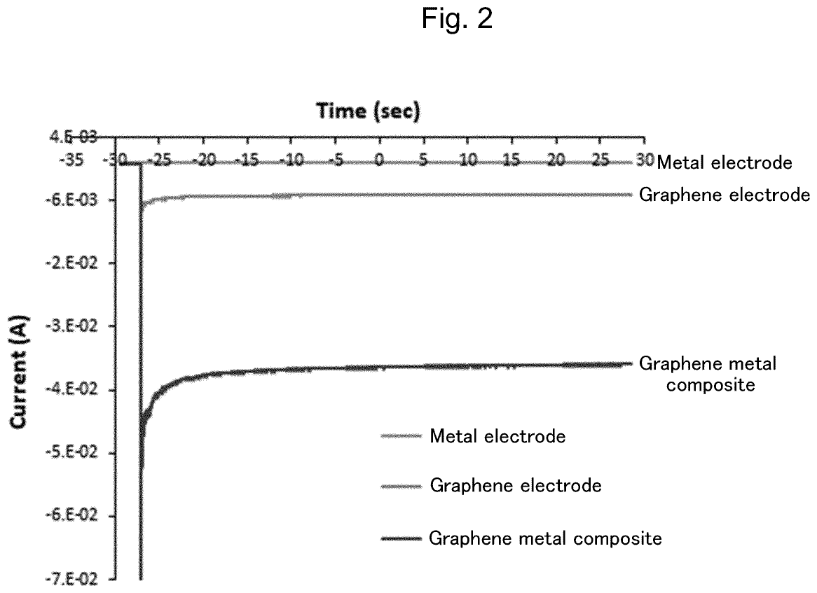

FIG. 2 is a graph showing comparing electrical conductivity characteristics of a hybrid graphene electrode (graphene metal composite), a metal electrode, and a graphene electrode according to the present invention. Compared to an electrode made of a gold (Au) thin film, the measured current signal of the graphene electrode becomes larger.

This is because the surface area of the electrode is large due to the porous structure and the inflow and emission of electrons through graphene is excellent, resulting in a larger flow of electrons generated in an electrochemical reaction.

In the case of a graphene metal composite electrode, the electrode has all the advantages of a graphene electrode, but the metal particles improve conductivity, making the resistance of the electrode very low.

Therefore, when measuring electrochemical signals using three types of electrodes, the largest current signal is generated when measured by the graphene metal composite electrode.

At this time, the sensitivity is better as the absolute value of the measured current signal is larger. Therefore, the graphene-metal composite electrode of the present invention has a very large signal to noise ratio (SNR) of a signal generated by using the advantages of the large surface area of graphene, the characteristics of more efficiently generating electrochemical reactions by absorption and emission of electrons, and the low resistance of metal particles to detect even low concentrations of target materials.

This technology may obtain 3D porous graphene through the photochemical and photothermal reactions. This technology has the advantage of being able to produce and pattern 3D graphene in a single step without a wet chemical step.

In addition, the silver (Ag) micro particles of the present invention may be used by coating the surface of copper metal with silver (Ag). Silver particles have excellent conductivity, but when considering cost, etc., the particles may be a desirable structure considering that the surface of the particle contributes greatly to conductivity even when used as a coating.

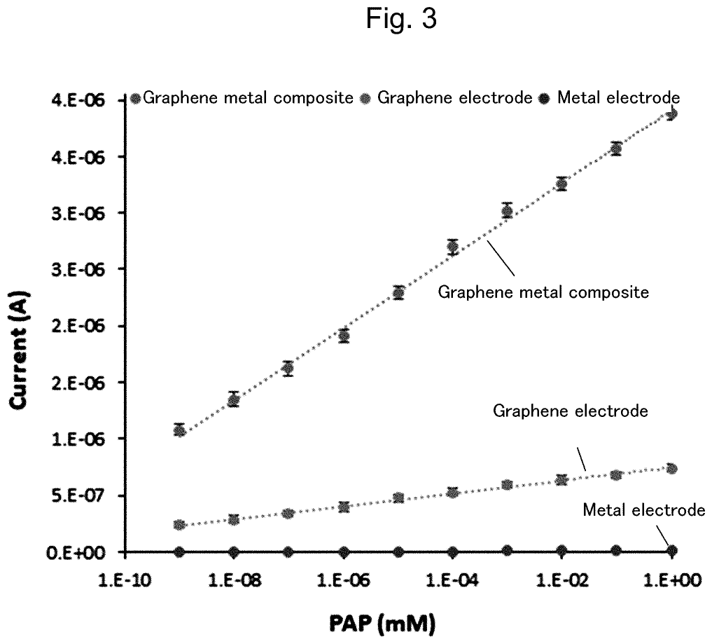

FIG. 3 is a graph showing measured currents according to a concentration of electrochemical measuring material (PAP) of a hybrid graphene electrode, a graphene electrode, and a metal electrode according to the present invention. For each electrode, the size of the current signal gradually increases depending on a PAP concentration. Further, it may be seen that with respect of the PAP of the same concentration, the signal of the graphene electrode, which has the advantages of surface area and electron inflow and emission, is larger than that of the metal electrode, and a signal of the graphene metal composite electrode, which has lower resistance than the graphene electrode, is measured larger.

The hybrid graphene electrode of the present invention may produce an interdigitated electrode (IDA) using a graphene metal composite material. The hybrid graphene electrode may be used as an electrochemical sensor that detects specific target materials using electrochemical reactions in the graphene metal composite.

The interdigitated electrode (IDA) has the characteristic of generating a current while electrons are moved between two finger-shaped electrodes through an electrochemical reaction. Ultrasensitive electrochemical detection of Alzheimer's disease may be achieved by using electrochemical enzyme-linked immunospecific assay (ELISA) measurements using the interdigitated electrode (IDA).

The US National Institute of Aging and Alzheimer Association (NIA-AA) proposed Alzheimer's biomarkers such as amyloid beta (Aβ) Aβ-40 and Aβ-42 in the brain and cerebrospinal fluid and total tau protein (t-tau) and phosphorylated tau protein (p-tau) reflecting neuronal cell injury. To electrochemically measure Aβ-42 and Aβ-40 and t-tau and p-tau, alkaline phosphatase (AP) is commonly used as an enzyme label for ELISA. The AP is attached to a second antibody, and thus the more Alzheimer's biomarkers are, the more AP enzymes are immobilized, thereby generating a larger electrochemical signal. An electroactive enzyme-substrate, p-amino phenylphosphate (PAPP) is produced in a chemical reaction with an enzyme product to produce an electroactive product, p-amino phenol (PAP). The PAP is oxidized to p-quinone imine (PQI) on the surface of the MHG interdigitated electrode (IDA), and then the PQI is reduced to PAP to cause a redox cycle of PAP. As the concentration of the Alzheimer's biomarker increases, more AP enzymes are immobilized in the reaction chamber to increase the electrochemical signal.

Using the principles, the measurement of the electroactive product, p-amino phenol (PAP) is important in diagnosing early Alzheimer's, so that the hybrid graphene electrode of the present invention may be applied as an electrode capable of distinguishing a very small amount.

Therefore, it may be seen that the shape of the micro particles in the hybrid graphene electrode affects the sensitivity of the measurement of the electroactive product, p-amino phenol (PAP), and the spherical shape of the silver (Ag) microparticles has the best sensitivity.

In addition, as the concentration of PAP molecules increases, the redox cycle of PAP molecules also increases, and as a result, current increases linearly to current measured by the MHG interdigitated electrode (IDA).

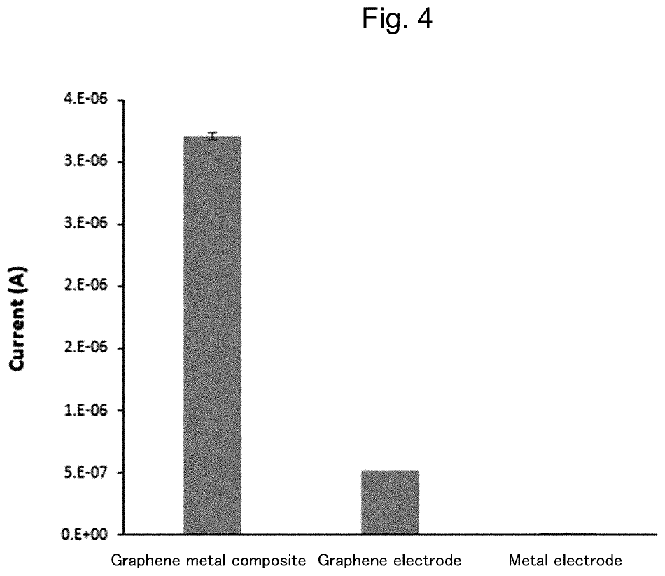

FIG. 4 is a diagram showing a difference in current signal measured for PAPs of the same concentration for the graphene metal composite electrode, the graphene electrode, and the metal electrode according to the present invention. It may be seen that the current signal of the graphene metal composite electrode of the present invention generates a larger signal than that of the comparison electrodes, and thus the signal to noise ratio (SNR) is greater than that of the comparison electrodes.

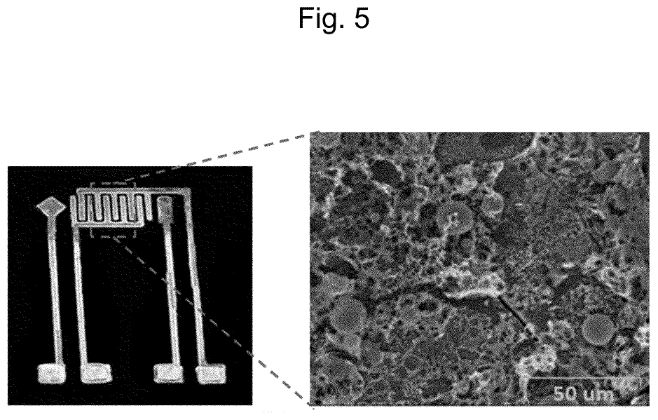

FIG. 5 is a diagram showing an interdigitated electrode (IDA) using a hybrid graphene electrode according to the present invention.

As described above, the present invention is not limited to the aforementioned embodiments and the accompanying drawings, and it will be obvious to those skilled in the technical field to which the present invention pertains that various substitutions, modifications, and changes may be made within the scope without departing from the technical spirit of the present invention.

1. A hybrid graphene electrode provided with a graphene composite having a structure in which a plurality of micro particles and multi-layered graphene are mixed, wherein the micro particles are metal or semiconductor particles and adhere to the surface or inside of the multi-layered graphene, some of the micro particles bond and coagulate with each other,
the multi-layered graphene has a three-dimensional structure in which several layers of graphene are laminated and bent in an arbitrary direction, and
a portion of empty spaces between the micro particles is filled with the multi-layered graphene, thus forming an interconnected structure, and electrons flow through the graphene composite.

2. The hybrid graphene electrode of claim 1, wherein graphene is coated on the surface of the micro particles.

3. The hybrid graphene electrode of claim 1, wherein the graphene composite is generated by photochemical or photothermal irradiation or heat treatment process.

4. The hybrid graphene electrode of claim 1, wherein external electrons are introduced or released through the graphene composite.

5. The hybrid graphene electrode of claim 1, wherein the micro particles include gold (Au), silicon (Si), silicon carbide (SiCx including Si₂C, SiC, or SiC₂), silicon oxide (SiOx including SiO or SiO₂), silver (Ag), and silver (Ag) coated on a copper metal surface.

6. The hybrid graphene electrode for an electrochemical sensor of claim 1, wherein in the graphene metal composite, a specific target material is detected by using an electrochemical reaction.

7. The hybrid graphene electrode of claim 1, wherein lithium (Li) ions are bonded to and separated from the graphene composite to cause charging and discharging.
